(19) United States
(12) Patent Application Publication
(10) Pub. No.: US 2012/0040410 A1
(43) Pub. Date: Feb. 16, 2012

(54) THERMOHEMICELLULASES FOR LIGNOCELLULOSIC DEGRADATION

(75) Inventors: Rolf A. Prade, Stillwater, OK (US); Fabio M. Squina, Sorocaba SP (BR)

(73) Assignee: THE BOARD OF REGENTS FOR OKLAHOMA STATE UNIVERSITY, Stillwater, OK (US)

(21) Appl. No.: 13/145,186

(22) PCT Filed: Jan. 19, 2010
(86) PCT No.: PCT/US10/21399

§ 371 Date: Sep. 19, 2011

Related U.S. Application Data
(60) Provisional application No. 61/145,665, filed on Jan. 19, 2009.

(51) Int. Cl. C12P 19/14; C12N 5/10; C07H 21/04; C12N 9/24

(52) U.S. Cl. 435/99; 435/200; 435/419; 536/23.2

(57) ABSTRACT

Hemicellulase (xylanase) enzymes possessing endo-xylanase, laminarase, mannanase, arabinase and arabinofuranosidase activity are useful to degrade hemicellulose and other substrates to their constituent sugars.

### STATEMENT OF FEDERALLY SPONSORED RESEARCH AND DEVELOPMENT

This invention was made with Government support under Grant No. DE-FG36-06GO16107, awarded by the U.S. Department of Energy. The United States government has certain rights in this invention.

### FIELD OF THE INVENTION

The invention generally relates to enzymes that are capable of hydrolyzing hemicellulose. In particular, the invention provides endo-xylanase, laminarase, mannanase, arabinase and arabinofuranosidase enzymes and methods of their use to hydrolyze hemicellulose.

### BACKGROUND OF THE INVENTION

Hemicellulose is the second most abundant biopolymer component of plant cell walls and contains a wealth of sugar residues. Due to their abundance and capacity for renewal, hemicelluloses have great potential for use in the production of many chemicals and materials, and especially for the production of biofuel. Hemicellulose is a branched biopolymer of D-xylose, linked by β-1,4-glucosyl linkages, arabinose and other attached sugars. Within plant cell walls, hemicellulose cross-links with pectin to form complex networks of polymeric compounds that include arabinans, galactomanans, laminarin and other polymers. In addition, sugars present on hemicellulosic polymers are often “decorated” with smaller molecules such as acetyl, methyl and ferulic acid, and these decorating side chains can interfere with the activity of enzymes that degrade the main polymers, impeding the extraction of sugars. Thus, esterases and ferulic acid esterases are important enzymes necessary for the degradation of hemicellulose.

When hemicellulose degrading enzymes have access to the polymeric region, they completely decompose the polymeric fraction and as a result, large quantities of xylose, arabinose and galacturonic acid are generated. Thus, a complete hemicellulosic-degrading enzymatic system consists of multiple enzymes such as xylanases, arabinases, arabinofuranosidases, laminarinases, mannanases and ferulic acid esterases. Depending on the specific composition of the plant cell wall polymers, cross-linking activities may also need to be considered. For example, in pectin rich plants it is also necessary to provide xyloglucanase and glucoronidase activity.

The hemicellulose backbone is constituted mainly of the pentosan xylose and can be broken down by xylanases. Cross-linking side chains are constituted of arabinan and galactomanan and can be broken down by arabinanases, galactanases and mannanases.

While some enzymes with these activities are known, the biotechnological use of biomass usually requires a high temperature environment for proper operation of the polymeric breakdown process. Therefore, only enzymes that can withstand high temperature conditions can be used efficiently. There is thus an ongoing need to discover, characterize, and make available thermophilic enzymes and enzyme systems that are capable of breaking down the components of hemicellulose and releasing the component sugars at high temperatures.

### SUMMARY OF THE INVENTION

Protein sequences which heretofore were not recognized as having enzymatic activity have been isolated and characterized as thermostable enzymes capable of degrading (hydrolyzing) hemicellulose at high temperatures. The enzymes, originating from various thermophilic bacteria, include an endo-xylanase, a laminarase, a mannanase, an arabinanase and an arabinofuranosidase. The activities displayed by the enzymes may be referred to herein as hemicellulase or hemicellulase-like activities, or, alternatively, as xylanase or xylanase-like activities. (The hemicellulose component of plant biomass is sometimes referred to as “xylan” due to the high proportion of xylose therein.) The enzymes are optimally catalytically active in the temperatures range of from about 70° C. to about 90° C. In some embodiments, the enzymes, or groups of enzymes (i.e. enzyme systems) comprising multiple thermostable catalytic activities may advantageously be used to degrade hemicellulose.

### BRIEF DESCRIPTION OF THE DRAWINGS

FIG. 1A-E. Amino acid sequences of: A, the endo-xylanase TH 1 (SEQ ID NO: 1); B, the laminarase TH 2 (SEQ ID NO: 2); C, the mannananse TH 3 (SEQ ID NO: 3); D, the arabinase TH 4 (SEQ ID NO: 4); E, the arabinofuranosidase TH 5 (SEQ ID NO: 5).

FIG. 2A-E. Exemplary nucleotide sequences encoding the enzymes of the invention. A, the endo-xylanase TH 1 (SEQ ID NO: 6); B, the laminarase TH 2 (SEQ ID NO: 7); C, the mannananse TH 3 (SEQ ID NO: 8); D, the arabinase TH 4 (SEQ ID NO: 9); E, the arabinofuranosidase TH 5 (SEQ ID NO: 10).

FIG. 3. Temperature, pH and thermostability profile for thermo hemicellulases. Two sets of enzymes were found TH1, TH2 and TH3 (Panels A, B and C) and TH4, TH5 (Panels A1, B1 and C1). Set 1 (TH1, TH2 and TH3) have a temperature optimum at 92° C., pH optimum at 6 and are thermostable at 90° C. to up to 30 hours while set 2 (TH4 and TH5) show a temperature optimum at 85° C., pH 6 and only TH4 is thermostable at 90° C.

### DETAILED DESCRIPTION

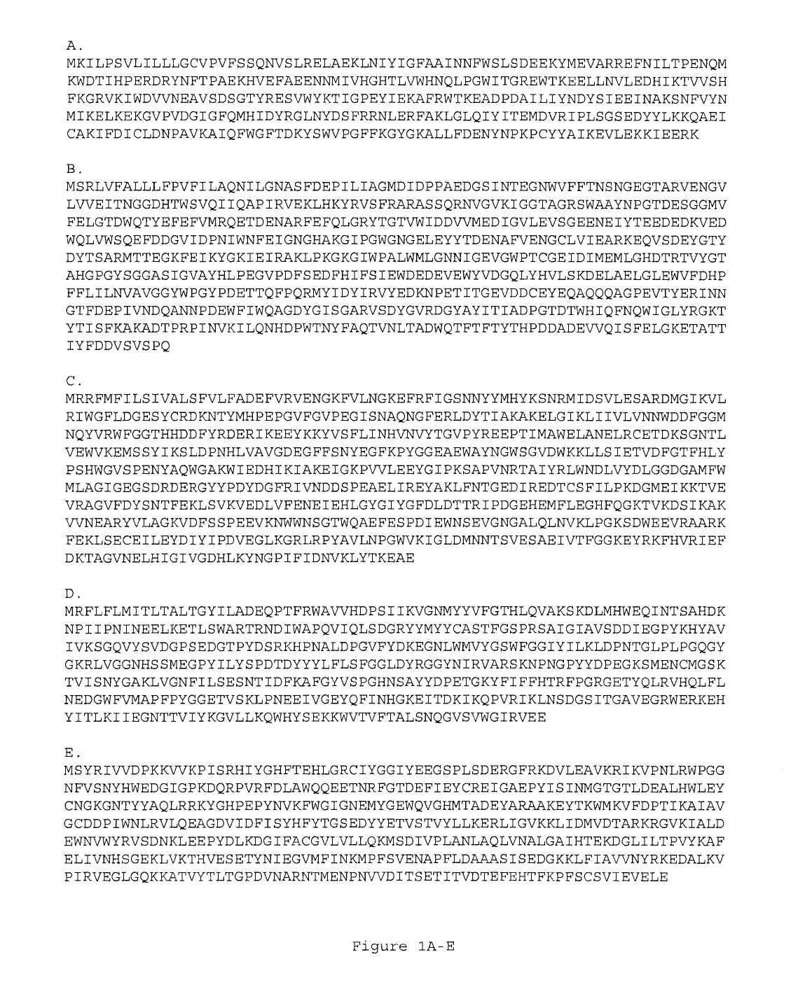

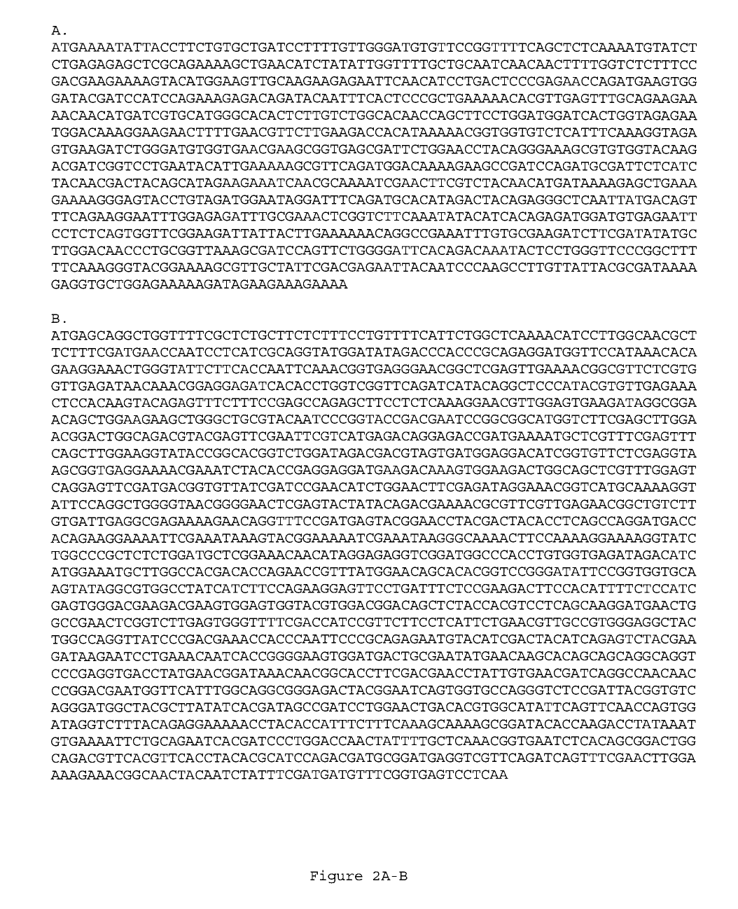

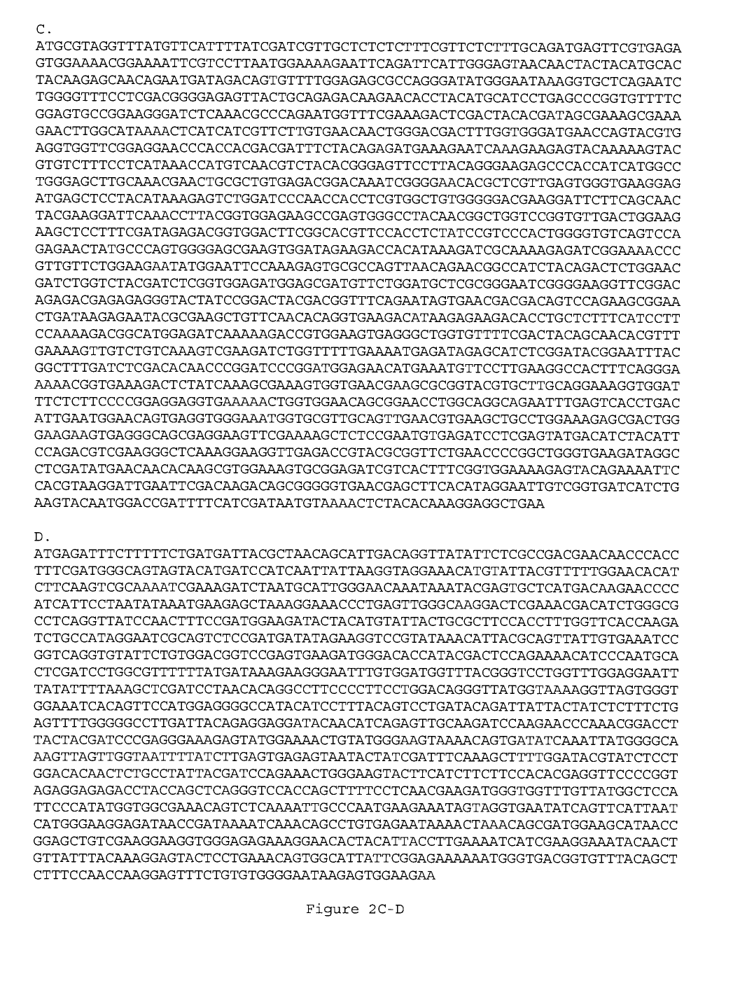

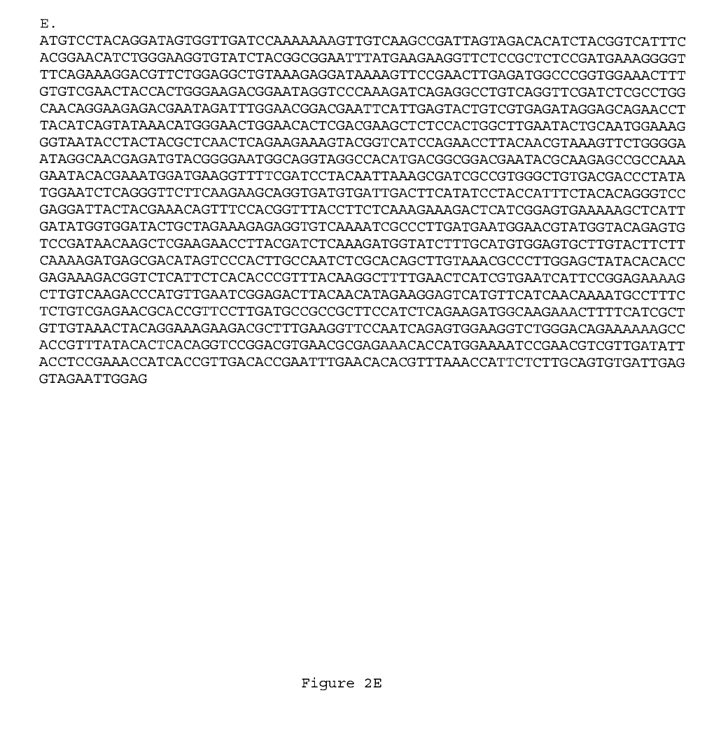

Thermostable hemicellulase (xylanase) enzymes capable of degrading (hydrolyzing) hemicellulose at high temperatures are disclosed herein. The hemicellulases include protein sequences that possess endo-xylanase, laminarase, mannanase, arabinase and arabinofuranosidase enzymatic activity. The amino acid sequences of the enzymes are presented in FIGS. 1A-E and exemplary nucleic acids encoding the enzymes are depicted in FIGS. 2A-E. Importantly, the enzymes are stable at high temperatures (e.g. in the range of from about 70° C. to about 90° C., depending on the particular enzyme). The enzymes are therefore suitable for use in catalyzing hemicellulose processing reactions, which are preferably carried out at high temperatures.

The enzymes may be used alone, i.e. one at a time in a hemicellulose degrading reaction, or, alternatively, two or more of the enzymes may be used together in such reactions. Thus, compositions comprising two or more of the enzymes are also provided. The compositions may include recombinant enzymes, or enzymes that have previously been isolated and substantially purified, and then combined into a mixture, or a mix of the two types of enzymes. Such a composition may also contain various factors that are useful or required for enzyme activity, e.g. buffering agents, cofactors, metal ions, etc. And the composition may be considered to include a substrate such as one or more hemicelluose-containing materials. This is especially useful since the enzymes described herein possess different substrate specificities and can thus carry out a multipronged or sequential breakdown of hemicellulose. In particular, the reaction catalyzed by the endo-xylanase is hydrolysis of beta 1,4 pentose glycosidic bonds resulting in smaller multimeric fragments; the reaction catalyzed by the laminarase is hydrolysis of mixed beta 1,4 and beta 1,3 glycosidic bonds resulting in smaller multimeric linear and branched fragments; the reaction catalyzed by the mannanase is hydrolysis of mannans, mannose-containing polysaccharides found in plants as storage material, in association with hemicellulose; the reaction catalyzed by the arabinase is hydrolysis of arabinan into smaller fragments and the reaction catalyzed by the arabinofuranosidase is hydrolysis of arabinofuranosyl residues from L-arabinose containing polysaccharides and hemicelluloses.

Prior to use, the enzymes of the invention may be prepared and isolated by standard methods. Similarly, optimal reaction conditions for each enzyme are determined, e.g. testing the ability of an enzyme to cleave a standard substrate under controlled conditions. Purified enzymes may be used, for example, to hydrolyze hemicellulose by contacting the hemicellullose with the enzyme under suitable reaction conditions, e.g. appropriate concentration, temperature, pH, medium, etc. Alternatively, individual enzymes may be used to catalyze the reaction for which they possess activity in non-hemicellulose substrates. For example, the arabinofuranosidase may be used to hydrolyze L-arabinose (L-arabinofuranose) containing polysaccharides from any source; the mannanase may be used to hydrolyze polyose chains containing mannose units (mannopolymers) such as glucamannan, galactoglucomannan and galactomannan from any source; the arabinofuranosidase enzyme may be used to hydrolyze arabinofuranosyl residues from L-arabinose containing polysaccharides from any source; etc.

The invention also contemplates the incorporation of nucleic acids encoding one or more of the enzymes into a host, for example, a plant, fungus, bacterium or animal. In the case where the host produces or is composed of hemicellulosic material (e.g., plants such as corn, switch grass, sugar cane, sorghum, pinus and eucalyptus), the host can be subjected to breakdown of the hemicellulosic material, for example, after harvest. That is, in a particular example, corn or switchgass transformed to include nucleic acids coding for the enzymes will express the enzymes internally, and after collection or harvest of the corn or switchgrass, the enzymes degrade the hemicellulose after preparation of the plant material in a suitable mileau and elevation of the temperature. Alternatively, the enzymes may be produced in or by the host cell and isolated and purified for use with another substrate.

Many types of hemicellulosic materials may be treated in accordance with this invention, including but not limited to lignocellulosic biomass such as agricultural residues (straws, hulls, stems, stalks), corn fiber, wood, municipal solid wastes (paper, cardboard, yard trash, and wood products), wastes from the pulp and paper industry, and herbaceous crops. Furthermore, the cellulose of many red algae contains a significant amount of mannose, e.g. the so-called α-cellulose from Porphyra is pure mannan. Such reactions may be carried out in order to obtain valuable breakdown products such as various fermentable sugars generated by hemicellulose catalysis. Alternatively, xylanases are also useful for various pretreatments of e.g. kraft pulp for other purposes such as for bleaching pulp that is used to make paper.

In addition, a variety of non-pulp applications exist for the enzymes. For example, the thermostable xylanase molecules of the present invention have a physiological temperature and pH optima such that they are useful as animal feeds additives since they can withstand the heat associated with feed sterilization and pellet formation, yet they exhibit optimal activity within an animal to aid in breakdown of ingested feed. Further, various xylanases have been reported to be useful in clarifying juice and wine; for extracting coffee, plant oils and starch; for the production of food thickeners; for altering texture in bakery products (e.g., to improve the quality of dough, to help bread rise); and for the processing of wheat and corn for starch production; as components of detergents and other cleaning compositions; etc.

Uses of particular enzymes include but are not limited to the following:

### Arabinanase

Specific applications of the enzyme include, but are not limited to, the saccharification of L-arabinose containing polysaccharides and hemicelluloses to fermentable sugars L-arabinose and xylose for subsequent fermentation to ethanol or arabitol; the treatment of plant materials for use as animal feed; delignification of pulp; hydrolysis of grape monoterpenyl glycosides during wine fermentation; and clarification and thinning of juices. Enzymes capable of degrading arabinans are becoming increasingly important to the food industry. In juice production, for example, the demand to increase yields in order to reduce production costs has necessitated the modification of traditional processes. The utilization of enzymatic pre-treatments of the fruit pulp before pressing with specific enzymatic products drastically improves the juice yield by solubilizing the cell wall polysaccharides.

### Arabinofuranosidase

Arabinofuranosidases have practical applications in various agro-industrial processes such as efficient conversion of hemicellulosic biomass to fuels and chemicals, delignification of pulp, efficient utilization of plant materials into animal feed, and hydrolysis of grape monoterpenyl glycosides during wine fermentation.

There is a growing need to discover suitable arabinofuranosidases for use in the conversion of hemicellulose to fermentable sugars for the subsequent production of fuel ethanol and other value-added chemicals. Arabinofuranosidases may be used in conjunction with xylanolytic enzymes for the treatment of hemicellulosic materials to produce fermentable sugars, particularly xylose and L-arabinose.

### Mannanase

Mannanase enzymes may be used in the commercial scale processing of mannans to simpler sugars for use in the food, feed, oil, paper, pulp, textile and biofuels industries.

### Xylanase

Xylanase enzymes maybe used in paper bleaching by removal of hemicellulose, and cross-linked lignin. Laminarase enzymes maybe used in hemicellulose pre-treatments adding the deconstruction of laminarin like polymers and guaranteeing access to the hemicellulose usually processed by endo-xylanase. Xylanases, laminarases, arabinases and arabinofuranosidases may also be used in combination as additives pretreatment processes of biomass, which intend the isolation of cellulose. Usually pre treatments involve high temperature aqueous reaction mixtures, a condition ideal for the enzymes described herein.

Exemplary amino acid sequences of the recombinant enzymes of the invention and exemplary nucleotide sequences that encode them are depicted in FIGS. 1A-E and 2A-E. However, those of skill in the art will recognize that the invention also encompasses variant proteins comprising amino acid sequences that are based on or derived from the sequences disclosed herein. By an amino acid sequence that is “derived from” or “based on” the sequence disclosed herein, we mean that a derived sequence (or variant sequence) displays at least about 50 to 100% identity to an amino acid sequence disclosed herein, or about 60 to 100% identify, or about 70 to 100% identity, or even from about 80 to 100% identity. In preferred embodiments, a variant sequence displays from about 90 to 100% or about 95 to 100% amino acid identity. In further preferred embodiments, a variant sequence is 95, 96, 97, 98 or 99% identical to at least one sequence disclosed herein. Variations in the sequences may be due to a number of factors and may include, for example: conservative or non-conservative amino acid substitutions; natural variations among different populations as isolated from natural sources; various deletions or insertions (which may be amino terminal, carboxyl terminal, or internal); addition of leader sequences to promote secretion from the cell; addition of targeting sequences to direct the intracellular destination of a polypeptide; etc. Such alterations may be naturally occurring or may be intentionally introduced (e.g. via genetic engineering) for any of a wide variety of reasons, e.g. in order to eliminate or introduce protease cleavage sites, to eliminate or introduce glycosylation sites, in order to improve solubility of the polypeptide, to facilitate polypeptide isolation (e.g. introduction of a histidine or other tag), as a result of a purposeful change in the nucleic acid sequence (see discussion of the nucleic acid sequence below) which results in a non-silent change in one or more codons and thus the translated amino acid, in order to improve thermal stability of the protein, etc. All such variant sequences (including without limitation fusion and/or chimeric proteins and other variations, modifications and derivatives) are encompassed by the present invention, so long as the resulting polypeptide is capable of catalyzing the enzyme activity of the original protein as disclosed herein (for example, at least about 50, 55, 60, 65, 70, 75, 80, 85, 90, 95, or 100%, or even more) of the activity of the protein as disclosed herein, at temperatures exceeding 70° C. For example, the invention includes shorter portions of the sequences that also retain the catalytic activity of the enzyme. The full-length protein sequences and/or active portions thereof are both referred to as polypeptides herein. In addition, the invention also includes chimeric or fusion proteins that include, for example: more than one of the enzymes disclosed herein (or active portions thereof); or one or more of the enzymes disclosed herein (or portions thereof) plus some other useful protein or peptide sequence(s), e.g. signal sequences, spacer or linker sequences, etc.

The invention also comprehends nucleic acid sequences that encode the proteins and polypeptides of the invention. Several exemplary nucleic acid sequences are provided herein. However, as is well known, due to the degeneracy of the nucleic acid triplet code, many other nucleic acid sequences that would encode an identical polypeptide could also be designed, and the invention also encompasses such nucleic acid sequences. Further, as described above, many useful variant forms of the proteins and peptides of the invention also exist, and nucleic acid sequences encoding such variants are intended to be encompassed by the present invention. In addition, such nucleic acid sequences may be varied for any of a variety of reasons, for example, to facilitate cloning, to facilitate transfer of a clone from one construct to another, to increase transcription or translation in a particular host cell (e.g. the sequences may be optimized for expression in, for example, corn, rice, yeast or other hosts), to add or replace promoter sequences, to add or eliminate a restriction cleavage site, etc. In addition, all genera of nucleic acids (e.g. DNA, RNA, various composite and hybrid nucleic acids, etc.) encoding proteins of the invention (or active portions thereof) are intended to be encompassed by the invention. Nucleic acid sequences encompassed by the invention generally include those which are from about 90% to about 100% homologous to the sequences disclosed herein, e.g. about 91, 92, 93, 94, 95, 96, 97, 98 or 99% homologous, as determined by methods that are known in the art.

The invention further comprehends vectors, which contain nucleic acid sequences encoding the polypeptides of the invention. Those of skill in the art are familiar with the many types of vectors, which can be useful for such a purpose, for example: plasmids, cosmids, various expression vectors, viral vectors, etc.

Production of the nucleic acids and proteins of the invention can be accomplished in any of many ways that are known to those of skill in the art. The sequences may be obtained and isolated and purified from a natural source. The sequences may be synthesized chemically using methods that are well-known to those of skill in the art. Alternatively, nucleotide sequences may be cloned using, for example, polymerase chain reaction (PCR) and/or other known molecular biology and genetic engineering techniques, and used to make (e.g. express or over-express) recombinant proteins. Recombinant proteins may be made from a plasmid contained within a bacterial host such as Escherichia coli, in insect expression systems, yeast expression systems, plant cell expression systems, etc. Further, the nucleic acid sequences may be optimized for expression in a particular organism or system. To that end, the present invention also encompasses a host cell that has been transformed (e.g. a transformed host cell) or otherwise manipulated to contain nucleic acids encoding the proteins and polypeptides of the invention, either as extra-chromosomal elements, or incorporated into the chromosome of the host. In particular, in the practice of the present invention, nucleic acid sequences encoding one or more of the hemicellulases may be introduced into plant cells, seeds, etc., to generate recombinant, transformed plants that contain the nucleic acids.

Plant transformation to incorporate one or more nucleic acids coding for one or more hemicellulase enzymes as described herein can be accomplished by a variety of techniques known to those of skill in the art. Plant transformation is the introduction of a foreign piece of DNA, conferring a specific trait, into host plant cell or tissue. Plant transformation can be carried out in a number of different ways depending on the species of plant in question. A number of mechanisms are available to transfer DNA into plant cells, examples of which include but are not limited to:

Agrobacterium mediated transformation is the easiest and simplest plant transformation technique. Plant tissue (often leaves) is cut in small pieces, and soaked in a fluid containing suspended Agrobacterium. Some cells along the cut will be transformed by the bacterium, which inserts its DNA into the cell. Placed on selectable rooting and shooting media, the plants will regrow. Some plants species can be transformed just by dipping the flowers into suspension of Agrobacterium and then planting the seeds in a selective medium.

Particle bombardment: Small gold or tungsten particles are coated with DNA and shot into young plant cells or plant embryos. Some genetic material will stay in the cells and transform them. The transformation efficiency is lower than in bacterial mediated transformation, but most plants can be transformed with this method.

Electroporation: Makes transient holes in cell membranes using brief electric shock. Plasmid DNA can enter the cell through these holes. This method is amenable to use with large plasmid DNA. Natural membrane-repair mechanisms will rapidly close the holes after the shock.

Viral transformation (transduction): The desired genetic material is packaged into a suitable plant virus and the modified virus is allowed to infect the plant. If the genetic material is DNA, it can recombine with the chromosomes to produce transformant cells. However, genomes of most plant viruses consist of single stranded RNA which replicates in the cytoplasm of infected cell. For such genomes this method is a form of transfection and not a real transformation, since the inserted genes never reach the nucleus of the cell and do not integrate into the host genome. The progeny of the infected plants is virus free and also free of the inserted gene.

Suitable examples of plants that may be transformed to include one or more hemicellulase enzymes or sets of enzymes include but are not limited to rice, corn, various grasses such as switchgrass, sugar cane, sorghum, pinus and eucalyptus, etc. Advantages of genetically engineering plants to contain and express the cellulase genes include but are not limited to the availability of the enzymes within the cell, ready to be activated by high temperatures, e.g. after the plant is harvested.

In some embodiments of the invention, the enzymes are produced and isolated from cultures of either a natural source (e.g. the bacterium in which they were identified) or from cultures of a host organism that has been genetically manipulated to contain and express nucleic acid sequences that encode the enzymes. Many such expression systems are known, e.g. those employing Escherichia coli, various Baculovirus systems, etc. Methods of purifying enzymes are also generally known. The enzymes may be employed as single isolated enzymes or as combinations of isolated enzymes. Further, in some cases it is not necessary to isolate the enzymes from the host cell in which they are produced. Instead, the host cell may be cultured together in a mixture that contains one or more suitable substrates for the enzymes, and the enzymes may be secreted directly into the mixture.

The hemicellulases of the invention have very high temperature optima, an optimal temperature being the temperature at which an enzyme is maximally active, as determined by a standard assay recognized by those of skill in the art. As described in the Examples section below, the lowest temperature optimum for an enzyme of the invention is about 68° C., and the highest temperature optimum is about 92° C. Further, the enzymes of the invention are thermally stable, i.e. they are capable of retaining catalytic activity at high temperatures (e.g. at their temperature maximum, or at temperatures that deviate somewhat from the maximum) for extended periods of time, for example, for at least for several hours (e.g. 1-24 hours), and in many cases, for several days (e.g. from 1-7 days or even longer). By “retain catalytic activity” we mean that the enzyme retains at least about 10, 20, 30, 40 or 50% or more of the activity displayed at the beginning of the extended time period, when measured under standard conditions; and preferably the enzyme retains 60, 65, 70, 75, 80, 85, 90, 95, or even 100% of the activity displayed at the beginning of the extended time period.

The enzymes of the invention are generally employed in reactions that are carried out at temperatures at or near those which are optimal for their activity. Some enzymes may be used over a wide temperature range (e.g. at a temperature that is about 50, 40, 30, 20, 10, 5 or fewer degrees lower than (below) the temperature optimum, and up to about 5, 10, 15, or more degrees greater than (above) the temperature optimum. For other enzymes, the range may be more restricted, i.e. they may display catalytic activity within a narrower temperature range of only less than about 10, or less than about 5, or fewer degrees of their optimal catalytic temperature. When carrying out a digestion reaction, the enzymes may be used one at a time sequentially (i.e. one enzyme is added, reaction occurs, and then another enzyme is added, with or without removal of the previous enzyme, and so on), or the reaction mixture may contain two or more of the enzymes (as an enzyme system) at the same time. When designing groups of enzymes to be included in an enzyme system, a suitable temperature at which all enzymes in the group are active will be selected as the temperature for reaction. For example, for the enzymes disclosed herein, the range of temperatures will be from about 70 to about 90° C. If an enzyme is used individually, the reaction may be carried out at a temperature near its optimum, or at which the enzyme retains sufficient activity to be useful. In addition, the selection of a reaction temperature may be based on other considerations, e.g. safety or other practical considerations of high temperature operations, or concerns about the cost of keeping a reaction mixture at a high temperature, the temperature used for preparing biomass for the reaction, the temperature of procedures that follow the reaction, etc.

The breakdown of hemicellulose may or may not be complete, depending on the desired endproducts, and the precise activity of the enzyme or enzymes that are used to carry out the process. Any desired grouping of the enzymes of the invention may be utilized to generate any desired endproduct that the enzymes are capable of producing from a suitable substrate. Further, one or more of the enzymes of the invention may be used in combination with other enzymes such as cellulases, or with enzymes having other types of activities. In one embodiment of the invention, a “system” could further include a yeast or other organism capable of fermenting sugars produced by the enzymes, e.g. to produce ethanol or other valuable fermentation products.

The invention also provides methods of use of the enzymes disclosed herein. Such methods generally involve combining one or more of the enzymes with a suitable substrate under conditions that allow, promote or result in catalysis of the substrate by the enzyme(s). Generally, the reaction will be carried out at a temperature in the range of from about 70 to about 90° C., and the length of time for a reaction will be in the range of from about one hour to about six days. Reactions are carried out in media such as aqueous media buffered to a suitable pH, e.g. in the range of from about pH 4 to about pH 9. Thereafter, the desired products (e.g. saccharides, bleached or treated pulp, etc.) may be harvested from the broth, or the reaction products may be further processed. For example, for the production of ethanol, fermentation of sugars in the broth may be carried out by known conventional batch or continuous fermentation processes, usually using yeast. Ethanol may be recovered by known stripping or extractive distillation processes.

The invention is further illustrated by the non-limiting examples provided below.

### EXAMPLES

### Example 1

Hemicellulose is the second most abundant biopolymer component of plant cell walls and often is decorated with smaller molecules such as acetyl, methyl and ferulic acid. Decorating side chains interfere with the activity of enzymes that degrade the main polymers (extraction of sugars). When hemicellulose-degrading enzymes have access to the polymeric region, they completely decompose the polymeric fraction and as a result large quantities of xylose, arabinose and galacturonic acid are generated. These sugars are substrates for ethanol (biofuel) production. At high temperatures most of the recalcitrant biomass polymers become enzymatically accessible, enabling enzymatic degradation of raw biomass. Here we describe a high-temperature operating thermo-stable cellulose enzyme system, consisting of xylanase, arabinase, arabinofuranosidase, laminarase and mannanase that hydrolyze internal glycosidic bonds releasing sugars and remove decorating features. The consolidated enzyme system operates optimally at temperatures above 85° C. and retain >85% of its enzymatic activity after a 30 hour incubation at 90° C.

### Isolation and Characterization of High-Temperature Operating and Thermo-Stable Hemicelluloses

A series of five high-temperature operating and thermo-stable hemicellulases-endo-xylanase, laminarinase, mannanase, arabinanase and an arabinofuranosidase-were identified from a genome wide bioinformatics screen (Table I). The corresponding genes were genetically manipulated adapting its expression to a laboratory tractable system (E. coli) by usage of a controlled promoter. Individual proteins were expressed and isolated (purified) from E. coli crude extracts and analyzed for activity and other physical and chemical properties. Table I describes the five enzymes isolated in this study.

TABLE I
Physical properties of the novel hemicellulases

| | Physical Properties | | | | | | |
|---|---|---|---|---|---|---|---|
| | | | | | Charge | Temperature | |
| Protein | Locus | CaZy | MW (d) | pI | (pH 7) | Optimum ° C. | pH Range |
| TH 1 | Tpet0854 | GH10 | 40,762 | 5.90 | −6.20 | 92 | 5-9 |
| TH 2 | Tpet0899 | GH16 | 72,610 | 4.37 | −66.10 | 87 | 5-9 |
| TH 3 | Tpet1542 | GH5 | 76,705 | 5.05 | −31.40 | 87 | 4-9 |
| TH 4 | Tpet0637 | GH43 | 53,463 | 6.86 | −1.00 | 77 | 5-6 |
| TH 5 | Tpet0631 | GH51 | 55,281 | 5.51 | −13.30 | 68 | 5-6 |

TH1 xylanase is active on wheat arabinoxylan but is not active on other substrates such as laminarin, sugar beet arabinan, debranched arabinan or gum locust bean. Conversely, TH2 laminarinase is only active on laminarin, TH3 mannanase on gum locust bean, TH4 arabinase on wheat arabinoxylan, sugar beet arabinan and debranched arabinan and TH5 arabinofuranosidase is active on sugar beet arabinan and debranched arabinan (Table II).

TABLE II
Substrate specific hemicellulase activity

| | Specific Activity (U) | | | | |
|---|---|---|---|---|---|
| Protein | WAX | LAM | SB | DB | GLB |
| TH 1 | 130 | nd | nd | nd | nd |
| TH 2 | nd | 111 | nd | nd | nd |
| TH 3 | nd | nd | nd | nd | 111 |
| TH 4 | 125 | nd | 11 | 100 | nd |
| TH 5 | nd | nd | 54 | 17 | nd |

WAX, wheat arabinoxylan;
LAM, laminarin from Laminara digitata;
SB, Arabinan (sugar beet);
DA, Debranched Arabinan Specific activity;
GLB, locust Bean.
Specific Activity, mM reducing sugar/mg protein/hour at 80° C., pH 6; 0.2% of specific substrate

### Mode of Operation

Table III shows the optimum temperature of operation of all five thermo-hemicellulases. The highest optimum was found for TH1 xylanase with and optimum of 92° C. and the lowest optimum was for TH5 arabinofuranosidase with 68° C. At 55° C. all THs lost at least 31% of their activity, and at 25° C. THs operate with less than 10% of their optimum activity.

TABLE III
High temperature profile of hemicellulases

| | Specific Activity | | | | |
|---|---|---|---|---|---|
| | | Temperature | % Activity at | | |
| Protein | U | optimum ° C. | 55° C. | 42° C. | 25° C. |
| TH 1 | 141.8 | 92.0 | 54.2 | 40.2 | 10.8 |
| TH 2 | 120.8 | 87.0 | 48.0 | 15.0 | 1.2 |
| TH 3 | 139.7 | 87.0 | 31.0 | 15.0 | 2.5 |
| TH 4 | 118.4 | 77.0 | 32.2 | 6.1 | 2.5 |
| TH 5 | 57.8 | 68.0 | 80.0 | 10.5 | 1.3 |

Specific Activity, mM reducing sugar/mg protein/hour at pH 6; 0.2% of specific substrate

### Thermo Stability

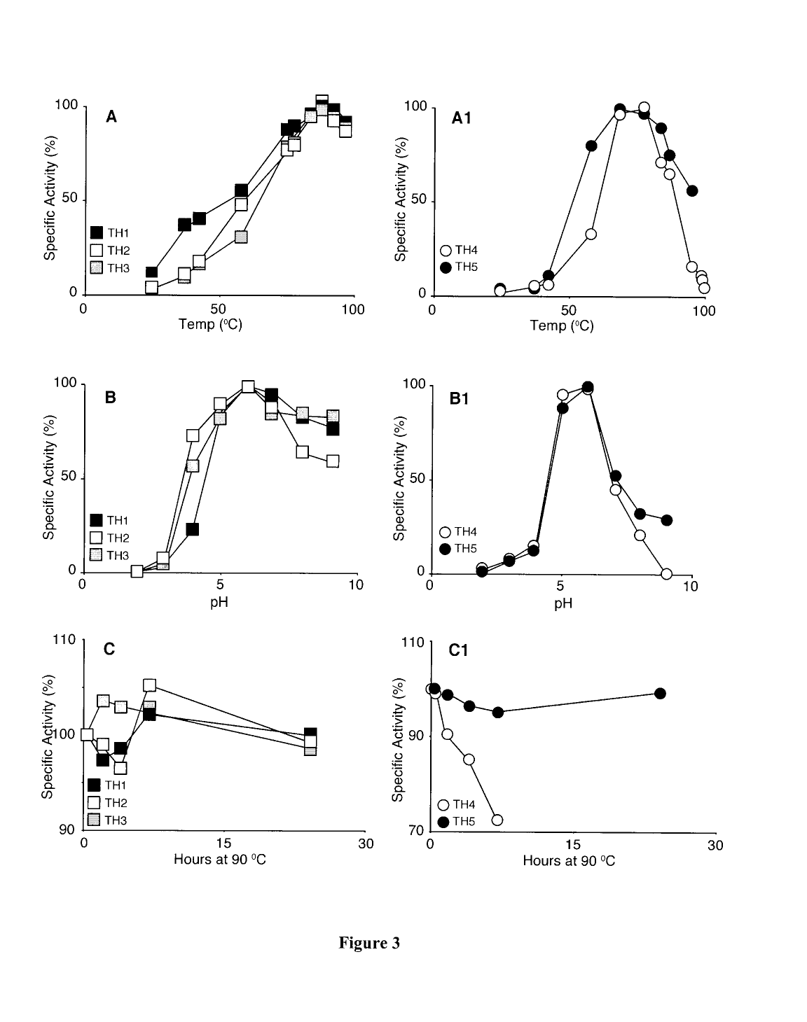

Thermostability at elevated temperatures is an important trait that thermo hemicellulases should exhibit because of their potential application in biomass pre-treatments that usually involve high temperature treatments. Thus, we have assayed our enzymes for stability at 90° C. and for the exception of one enzyme (TH5) all retain at least 90% of the original activity after a 30 hr incubation period (FIG. 3).

### Modes of Use

Pretreatment of biomass to enhance access to recalcitrant cellulose most of the time involves heating of crude biomass (e.g., sugar cane bagasse, corn stover, switch grass etc) at high temperatures in an acidic or alkaline environment in order to loosen cellulosic fibers and make them more accessible to cellulases. However, one third of all biomass is composed of hemicellulose polymers (xylans and cross-linked pectin), which represent a rich source of fermentable sugars if broken down into monomers, mainly xylose, arabinose and galacturonic acid. Thus, enhancing existing high temperature pre-treatments with thermostable hemicellulases has the benefit of extracting these fermentable sugars useful for further processing such as production of the biofuel ethanol.

Moreover, there are a number of applications in which the removal of hemicellulases without affecting the cellulosic fraction of biomass is not only beneficial but essential. For example the recovery of cellulosic fibers to manufacture high-quality paper or fibers useful in the textile industry (flax, linen, ramie etc) requires the complete removal of lignin's, pectin's and other hemicellulosic polymers. Thus, one aspect of the invention is to provide an improvement in methods of biofuel production.

### Methods

Cloning Genomic DNA of Thermotoga petrophila RKU-1 served as PCR template for the cloning of TH1, TH2, TH3, Th4 and TH5. Primer sequences are shown in Table IV.

TABLE IV
Oligonucleotide sequences used in this study.

| Primer | Sequence (5′ → 3′) | SEQ ID NO: |
|---|---|---|
| TH1_fw | AAAATATTACCTTCTGTGCTGATC | 11 |
| TH1_rv | CTGGAGAAAAAGATAGAAGAAAGAAAA | 12 |
| TH2_fw | AGCAGGCTGGTTTTCGCT | 13 |
| TH2_rv | CGATGATGTTTCGGTGAGTCCTCAA | 14 |
| TH3_fw | CGTAGGTTTATGTTCATTTTATCGATC | 15 |
| TH3_rv | TCTACACAAAGGAGGCTGAA | 16 |
| TH4_fw | AGATTTCTTTTTCTGATGATTACGC | 17 |
| TH4_rv | TGGGGAATAAGAGTGGAAGAA | 18 |
| TH5_fw | TCCTACAGGATAGTGGTTGATCC | 19 |
| TH5_rv | CAGTGTGATTGAGGTAGAATTGGAG | 20 |

Restriction sites were introduced (bold letters). All gene segments generated were cloned into the NcoI and XbaI sites of pBAD/Myc-His vector (Invitrogen), which carries a fusion sequence encoding six histidine residues at the C-terminus of expressed proteins. The expression plasmids were used to transform Escherichia coli TOP 10F′ (Invitrogen). All constructs were verified by DNA sequencing.

### Expression and Purification

An overnight growth of transformed E. coli strain containing the fusion protein vector was inoculated into fresh Luria-Bertani medium containing ampicillin. When the OD⁶⁰⁰ reached 0.5-0.6, L-arabinose was added to a final concentration of 0.2%. The culture was allowed to grow for another 4-5 h at 37° C. and the cells were collected by centrifugation. The pellet was stored at −80° C. prior to further processes. Cells were disrupted by sonication and the cell debris was removed by centrifugation at 10,000×g for 20 min. The protein pool was then heat treated at 95° C. for 5 min, and denatured proteins were removed by centrifugation at 12,000×g for 20 min. The recombinant protein carrying a His6 tag was then purified by immobilized-metal-chelate affinity chromatography (Qiagen).

### Hydrolysis of Hemicellulose and Hemicellulose Derivatives (Pectin and Cross Linking Polymers)

Hydrolysis of wheat (beech wood and oat spelt) arabinoxylan, laminarin, arabinan and debranched arabinan was measured spectrophotometrically by the increase of reducing ends at various temperatures and pH. The amount of reducing sugar ends was determined by the dinitrosalicyclic acid (DNS) method. The assay mix contained 10 μl of diluted enzymes, 30 μl of 100 mm sodium phosphate buffer, pH 6.0, and 20 μl of 0.5% (wt/vol) soluble substrates for 5 minutes. The reaction was terminated by adding 60 μl of DNS Solution. The absorbance of assay mix was read at 575 nm after the incubation at 95° C. for 5 min. The activity of enzymes as a function of temperature and pH was measured with the specific polysaccharide substrate. Temperature gradient was achieved using PCR cycler (MJ Research). Phosphate/citrate buffers were used to generate pH gradient (i.e., 2, 3, 4, 5, 6, 7, 8, 9.1). For thermostability assay, enzyme was incubated at 90° C. After aliquot of enzymes was taken, the residual activity was measured with the specific polysaccharide substrate.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A recombinant or isolated and purified heat stable endo-xylanase having an amino acid sequence as set forth in SEQ ID NO: 1.

2. A recombinant or isolated and purified heat stable laminarase having an amino acid sequence as set forth in SEQ ID NO: 2.

3. A recombinant or isolated and purified heat stable mannanase having an amino acid sequence as set forth in SEQ ID NO: 3.

4. A recombinant or isolated and purified heat stable arabinase having an amino acid sequence as set forth in SEQ ID NO: 4.

5. A recombinant or isolated and purified heat stable arabinofuranosidase having an amino acid sequence as set forth in SEQ ID NO: 5.

6. A transformed host cell expressing one or more recombinant enzymes with an amino acid sequence represented by an amino acid sequence selected from the group consisting of SEQ ID NOS: 1-5.

7. The transformed host cell of claim 6, wherein said transformed host cell is a plant cell.

8. A recombinant nucleic acid sequence comprising SEQ ID NO: 6.

9. A recombinant nucleic acid sequence comprising SEQ ID NO: 7.

10. A recombinant nucleic acid sequence comprising SEQ ID NO: 8.

11. A recombinant nucleic acid sequence comprising SEQ ID NO: 9.

12. A recombinant nucleic acid sequence comprising SEQ ID NO: 10.

13. A method of breaking down hemicellulose, comprising the steps of
contacting said hemicellulose with one or more enzymes selected from:
a recombinant or isolated and purified heat stable endo-xylanase having an amino acid sequence as set forth in SEQ ID NO: 1;
a recombinant or isolated and purified heat stable laminarase having an amino acid sequence as set forth in SEQ ID NO: 2;
a recombinant or isolated and purified heat stable mannanase having an amino acid sequence as set forth in SEQ ID NO: 3;
a recombinant or isolated and purified heat stable arabinase having an amino acid sequence as set forth in SEQ ID NO: 4; and
a recombinant or isolated and purified heat stable arabinofuranosidase having an amino acid sequence as set forth in SEQ ID NO: 5.

14. In a biofuel production method, the improvement comprising
breaking down hemicellulose by exposing a composition containing said hemicellulose to one or more enzymes, at least one of which has an amino acid sequence selected from SEQ ID NOS: 1-5.


SEQUENCE LISTING (20 sequences)

SEQ ID NO: 1 - protein, 347 aa, Thermotoga petrophila
MKILPSVLILLLGCVPVFSSQNVSLRELAEKLNIYIGFAAINNFWSLSDEEKYMEVARREFNILTPENQMKWDTIHPERDRYNFTPAEKHVEFAEENNMIVHGHTLVWHNQLPGWITGREWTKEELLNVLEDHIKTVVSHFKGRVKIWDVVNEAVSDSGTYRESVWYKTIGPEYIEKAFRWTKEADPDAILIYNDYSIEEINAKSNFVYNMIKELKEKGVPVDGIGFQMHIDYRGLNYDSFRRNLERFAKLGLQIYITEMDVRIPLSGSEDYYLKKQAEICAKIFDICLDNPAVKAIQFWGFTDKYSWVPGFFKGYGKALLFDENYNPKPCYYAIKEVLEKKIEERK

SEQ ID NO: 2 - protein, 641 aa, Thermotoga petrophila [sequence omitted]

SEQ ID NO: 3 - protein, 667 aa, Thermotoga petrophila
MRRFMFILSIVALSFVLFADEFVRVENGKFVLNGKEFRFIGSNNYYMHYKSNRMIDSVLESARDMGIKVLRIWGFLDGESYCRDKNTYMHPEPGVFGVPEGISNAQNGFERLDYTIAKAKELGIKLIIVLVNNWDDFGGMNQYVRWFGGTHHDDFYRDERIKEEYKKYVSFLINHVNVYTGVPYREEPTIMAWELANELRCETDKSGNTLVEWVKEMSSYIKSLDPNHLVAVGDEGFFSNYEGFKPYGGEAEWAYNGWSGVDWKKLLSIETVDFGTFHLYPSHWGVSPENYAQWGAKWIEDHIKIAKEIGKPVVLEEYGIPKSAPVNRTAIYRLWNDLVYDLGGDGAMFWMLAGIGEGSDRDERGYYPDYDGFRIVNDDSPEAELIREYAKLFNTGEDIREDTCSFILPKDGMEIKKTVEVRAGVFDYSNTFEKLSVKVEDLVFENEIEHLGYGIYGFDLDTTRIPDGEHEMFLEGHFQGKTVKDSIKAKVVNEARYVLAGKVDFSSPEEVKNWWNSGTWQAEFESPDIEWNSEVGNGALQLNVKLPGKSDWEEVRAARKFEKLSECEILEYDIYIPDVEGLKGRLRPYAVLNPGWVKIGLDMNNTSVESAEIVTFGGKEYRKFHVRIEFDKTAGVNELHIGIVGDHLKYNGPIFIDNVKLYTKEAE

SEQ ID NO: 4 - protein, 471 aa, Thermotoga petrophila [sequence omitted]

SEQ ID NO: 5 - protein, 484 aa, Thermotoga petrophila
MSYRIVVDPKKVVKPISRHIYGHFTEHLGRCIYGGIYEEGSPLSDERGFRKDVLEAVKRIKVPNLRWPGGNFVSNYHWEDGIGPKDQRPVRFDLAWQQEETNRFGTDEFIEYCREIGAEPYISINMGTGTLDEALHWLEYCNGKGNTYYAQLRRKYGHPEPYNVKFWGIGNEMYGEWQVGHMTADEYARAAKEYTKWMKVFDPTIKAIAVGCDDPIWNLRVLQEAGDVIDFISYHFYTGSEDYYETVSTVYLLKERLIGVKKLIDMVDTARKRGVKIALDEWNVWYRVSDNKLEEPYDLKDGIFACGVLVLLQKMSDIVPLANLAQLVNALGAIHTEKDGLILTPVYKAFELIVNHSGEKLVKTHVESETYNIEGVMFINKMPFSVENAPFLDAAASISEDGKKLFIAVVNYRKEDALKVPIRVEGLGQKKATVYTLTGPDVNARNTMENPNVVDITSETITVDTEFEHTFKPFSCSVIEVELE

SEQ ID NO: 6 - DNA, 1041 bp, Thermotoga petrophila
atgaaaatattaccttctgtgctgatccttttgttgggatgtgttccggttttcagctctcaaaatgtatctctgagagagctcgcagaaaagctgaacatctatattggttttgctgcaatcaacaacttttggtctctttccgacgaagaaaagtacatggaagttgcaagaagagaattcaacatcctgactcccgagaaccagatgaagtgggatacgatccatccagaaagagacagatacaatttcactcccgctgaaaaacacgttgagtttgcagaagaaaacaacatgatcgtgcatgggcacactcttgtctggcacaaccagcttcctggatggatcactggtagagaatggacaaaggaagaacttttgaacgttcttgaagaccacataaaaacggtggtgtctcatttcaaaggtagagtgaagatctgggatgtggtgaacgaagcggtgagcgattctggaacctacagggaaagcgtgtggtacaagacgatcggtcctgaatacattgaaaaagcgttcagatggacaaaagaagccgatccagatgcgattctcatctacaacgactacagcatagaagaaatcaacgcaaaatcgaacttcgtctacaacatgataaaagagctgaaagaaaagggagtacctgtagatggaataggatttcagatgcacatagactacagagggctcaattatgacagtttcagaaggaatttggagagatttgcgaaactcggtcttcaaatatacatcacagagatggatgtgagaattcctctcagtggttcggaagattattacttgaaaaaacaggccgaaatttgtgcgaagatcttcgatatatgcttggacaaccctgcggttaaagcgatccagttctggggattcacagacaaatactcctgggttcccggctttttcaaagggtacggaaaagcgttgctattcgacgagaattacaatcccaagccttgttattacgcgataaaagaggtgctggagaaaaagatagaagaaagaaaa

SEQ ID NO: 7 - DNA, 1923 bp, Thermotoga petrophila
atgagcaggctggttttcgctctgcttctctttcctgttttcattctggctcaaaacatccttggcaacgcttctttcgatgaaccaatcctcatcgcaggtatggatatagacccacccgcagaggatggttccataaacacagaaggaaactgggtattcttcaccaattcaaacggtgagggaacggctcgagttgaaaacggcgttctcgtggttgagataacaaacggaggagatcacacctggtcggttcagatcatacaggctcccatacgtgttgagaaactccacaagtacagagtttctttccgagccagagcttcctctcaaaggaacgttggagtgaagataggcggaacagctggaagaagctgggctgcgtacaatcccggtaccgacgaatccggcggcatggtcttcgagcttggaacggactggcagacgtacgagttcgaattcgtcatgagacaggagaccgatgaaaatgctcgtttcgagtttcagcttggaaggtataccggcacggtctggatagacgacgtagtgatggaggacatcggtgttctcgaggtaagcggtgaggaaaacgaaatctacaccgaggaggatgaagacaaagtggaagactggcagctcgtttggagtcaggagttcgatgacggtgttatcgatccgaacatctggaacttcgagataggaaacggtcatgcaaaaggtattccaggctggggtaacggggaactcgagtactatacagacgaaaacgcgttcgttgagaacggctgtcttgtgattgaggcgagaaaagaacaggtttccgatgagtacggaacctacgactacacctcagccaggatgaccacagaaggaaaattcgaaataaagtacggaaaaatcgaaataagggcaaaacttccaaaaggaaaaggtatctggcccgctctctggatgctcggaaacaacataggagaggtcggatggcccacctgtggtgagatagacatcatggaaatgcttggccacgacaccagaaccgtttatggaacagcacacggtccgggatattccggtggtgcaagtataggcgtggcctatcatcttccagaaggagttcctgatttctccgaagacttccacattttctccatcgagtgggacgaagacgaagtggagtggtacgtggacggacagctctaccacgtcctcagcaaggatgaactggccgaactcggtcttgagtgggttttcgaccatccgttcttcctcattctgaacgttgccgtgggaggctactggccaggttatcccgacgaaaccacccaattcccgcagagaatgtacatcgactacatcagagtctacgaagataagaatcctgaaacaatcaccggggaagtggatgactgcgaatatgaacaagcacagcagcaggcaggtcccgaggtgacctatgaacggataaacaacggcaccttcgacgaacctattgtgaacgatcaggccaacaacccggacgaatggttcatttggcaggcgggagactacggaatcagtggtgccagggtctccgattacggtgtcagggatggctacgcttatatcacgatagccgatcctggaactgacacgtggcatattcagttcaaccagtggataggtctttacagaggaaaaacctacaccatttctttcaaagcaaaagcggatacaccaagacctataaatgtgaaaattctgcagaatcacgatccctggaccaactattttgctcaaacggtgaatctcacagcggactggcagacgttcacgttcacctacacgcatccagacgatgcggatgaggtcgttcagatcagtttcgaacttggaaaagaaacggcaactacaatctatttcgatgatgtttcggtgagtcctcaa

SEQ ID NO: 8 - DNA, 2001 bp, Thermotoga petrophila
atgcgtaggtttatgttcattttatcgatcgttgctctctctttcgttctctttgcagatgagttcgtgagagtggaaaacggaaaattcgtccttaatggaaaagaattcagattcattgggagtaacaactactacatgcactacaagagcaacagaatgatagacagtgttttggagagcgccagggatatgggaataaaggtgctcagaatctggggtttcctcgacggggagagttactgcagagacaagaacacctacatgcatcctgagcccggtgttttcggagtgccggaagggatctcaaacgcccagaatggtttcgaaagactcgactacacgatagcgaaagcgaaagaacttggcataaaactcatcatcgttcttgtgaacaactgggacgactttggtgggatgaaccagtacgtgaggtggttcggaggaacccaccacgacgatttctacagagatgaaagaatcaaagaagagtacaaaaagtacgtgtctttcctcataaaccatgtcaacgtctacacgggagttccttacagggaagagcccaccatcatggcctgggagcttgcaaacgaactgcgctgtgagacggacaaatcggggaacacgctcgttgagtgggtgaaggagatgagctcctacataaagagtctggatcccaaccacctcgtggctgtgggggacgaaggattcttcagcaactacgaaggattcaaaccttacggtggagaagccgagtgggcctacaacggctggtccggtgttgactggaagaagctcctttcgatagagacggtggacttcggcacgttccacctctatccgtcccactggggtgtcagtccagagaactatgcccagtggggagcgaagtggatagaagaccacataaagatcgcaaaagagatcggaaaacccgttgttctggaagaatatggaattccaaagagtgcgccagttaacagaacggccatctacagactctggaacgatctggtctacgatctcggtggagatggagcgatgttctggatgctcgcgggaatcggggaaggttcggacagagacgagagagggtactatccggactacgacggtttcagaatagtgaacgacgacagtccagaagcggaactgataagagaatacgcgaagctgttcaacacaggtgaagacataagagaagacacctgctctttcatccttccaaaagacggcatggagatcaaaaagaccgtggaagtgagggctggtgttttcgactacagcaacacgtttgaaaagttgtctgtcaaagtcgaagatctggtttttgaaaatgagatagagcatctcggatacggaatttacggctttgatctcgacacaacccggatcccggatggagaacatgaaatgttccttgaaggccactttcagggaaaaacggtgaaagactctatcaaagcgaaagtggtgaacgaagcgcggtacgtgcttgcaggaaaggtggatttctcttccccggaggaggtgaaaaactggtggaacagcggaacctggcaggcagaatttgagtcacctgacattgaatggaacagtgaggtgggaaatggtgcgttgcagttgaacgtgaagctgcctggaaagagcgactgggaagaagtgagggcagcgaggaagttcgaaaagctctccgaatgtgagatcctcgagtatgacatctacattccagacgtcgaagggctcaaaggaaggttgagaccgtacgcggttctgaaccccggctgggtgaagataggcctcgatatgaacaacacaagcgtggaaagtgcggagatcgtcactttcggtggaaaagagtacagaaaattccacgtaaggattgaattcgacaagacagcgggggtgaacgagcttcacataggaattgtcggtgatcatctgaagtacaatggaccgattttcatcgataatgtaaaactctacacaaaggaggctgaa

SEQ ID NO: 9 - DNA, 1413 bp, Thermotoga petrophila
atgagatttctttttctgatgattacgctaacagcattgacaggttatattctcgccgacgaacaacccacctttcgatgggcagtagtacatgatccatcaattattaaggtaggaaacatgtattacgtttttggaacacatcttcaagtcgcaaaatcgaaagatctaatgcattgggaacaaataaatacgagtgctcatgacaagaaccccatcattcctaatataaatgaagagctaaaggaaaccctgagttgggcaaggactcgaaacgacatctgggcgcctcaggttatccaactttccgatggaagatactacatgtattactgcgcttccacctttggttcaccaagatctgccataggaatcgcagtctccgatgatatagaaggtccgtataaacattacgcagttattgtgaaatccggtcaggtgtattctgtggacggtccgagtgaagatgggacaccatacgactccagaaaacatcccaatgcactcgatcctggcgttttttatgataaagaagggaatttgtggatggtttacgggtcctggtttggaggaatttatattttaaagctcgatcctaacacaggccttccccttcctggacagggttatggtaaaaggttagtgggtggaaatcacagttccatggaggggccatacatcctttacagtcctgatacagattattactatctctttctgagttttgggggccttgattacagaggaggatacaacatcagagttgcaagatccaagaacccaaacggaccttactacgatcccgagggaaagagtatggaaaactgtatgggaagtaaaacagtgatatcaaattatggggcaaagttagttggtaattttatcttgagtgagagtaatactatcgatttcaaagcttttggatacgtatctcctggacacaactctgcctattacgatccagaaactgggaagtacttcatcttcttccacacgaggttccccggtagaggagagacctaccagctcagggtccaccagcttttcctcaacgaagatgggtggtttgttatggctccattcccatatggtggcgaaacagtctcaaaattgcccaatgaagaaatagtaggtgaatatcagttcattaatcatgggaaggagataaccgataaaatcaaacagcctgtgagaataaaactaaacagcgatggaagcataaccggagctgtcgaaggaaggtgggagagaaaggaacactacattaccttgaaaatcatcgaaggaaatacaactgttatttacaaaggagtactcctgaaacagtggcattattcggagaaaaaatgggtgacggtgtttacagctctttccaaccaaggagtttctgtgtggggaataagagtggaagaa

SEQ ID NO: 10 - DNA, 1452 bp, Thermotoga petrophila
atgtcctacaggatagtggttgatccaaaaaaagttgtcaagccgattagtagacacatctacggtcatttcacggaacatctgggaaggtgtatctacggcggaatttatgaagaaggttctccgctctccgatgaaaggggtttcagaaaggacgttctggaggctgtaaagaggataaaagttccgaacttgagatggcccggtggaaactttgtgtcgaactaccactgggaagacggaataggtcccaaagatcagaggcctgtcaggttcgatctcgcctggcaacaggaagagacgaatagatttggaacggacgaattcattgagtactgtcgtgagataggagcagaaccttacatcagtataaacatgggaactggaacactcgacgaagctctccactggcttgaatactgcaatggaaagggtaatacctactacgctcaactcagaagaaagtacggtcatccagaaccttacaacgtaaagttctggggaataggcaacgagatgtacggggaatggcaggtaggccacatgacggcggacgaatacgcaagagccgccaaagaatacacgaaatggatgaaggttttcgatcctacaattaaagcgatcgccgtgggctgtgacgaccctatatggaatctcagggttcttcaagaagcaggtgatgtgattgacttcatatcctaccatttctacacagggtccgaggattactacgaaacagtttccacggtttaccttctcaaagaaagactcatcggagtgaaaaagctcattgatatggtggatactgctagaaagagaggtgtcaaaatcgcccttgatgaatggaacgtatggtacagagtgtccgataacaagctcgaagaaccttacgatctcaaagatggtatctttgcatgtggagtgcttgtacttcttcaaaagatgagcgacatagtcccacttgccaatctcgcacagcttgtaaacgcccttggagctatacacaccgagaaagacggtctcattctcacacccgtttacaaggcttttgaactcatcgtgaatcattccggagaaaagcttgtcaagacccatgttgaatcggagacttacaacatagaaggagtcatgttcatcaacaaaatgcctttctctgtcgagaacgcaccgttccttgatgccgccgcttccatctcagaagatggcaagaaacttttcatcgctgttgtaaactacaggaaagaagacgctttgaaggttccaatcagagtggaaggtctgggacagaaaaaagccaccgtttatacactcacaggtccggacgtgaacgcgagaaacaccatggaaaatccgaacgtcgttgatattacctccgaaaccatcaccgttgacaccgaatttgaacacacgtttaaaccattctcttgcagtgtgattgaggtagaattggag

SEQ ID NO: 11 - DNA, 24 bp, Artificial Sequence
  Synthetic oligonucleotide primer
aaaatattaccttctgtgctgatc

SEQ ID NO: 12 - DNA, 27 bp, Artificial Sequence
  Synthetic oligonucleotide primer
ctggagaaaaagatagaagaaagaaaa

SEQ ID NO: 13 - DNA, 18 bp, Artificial Sequence
  Synthetic oligonucleotide primer
agcaggctggttttcgct

SEQ ID NO: 14 - DNA, 25 bp, Artificial Sequence
  Synthetic oligonucleotide primer
cgatgatgtttcggtgagtcctcaa

SEQ ID NO: 15 - DNA, 27 bp, Artificial Sequence
  Synthetic oligonucleotide primer
cgtaggtttatgttcattttatcgatc

SEQ ID NO: 16 - DNA, 20 bp, Artificial Sequence
  Synthetic oligonucleotide primer
tctacacaaaggaggctgaa

SEQ ID NO: 17 - DNA, 25 bp, Artificial Sequence
  Synthetic oligonucleotide primer
agatttctttttctgatgattacgc

SEQ ID NO: 18 - DNA, 21 bp, Artificial Sequence
  Synthetic oligonucleotide primer
tggggaataagagtggaagaa

SEQ ID NO: 19 - DNA, 23 bp, Artificial Sequence
  Synthetic oligonucleotide primer
tcctacaggatagtggttgatcc

SEQ ID NO: 20 - DNA, 25 bp, Artificial Sequence
  Synthetic oligonucleotide primer
cagtgtgattgaggtagaattggag